(19) United States
(12) Patent Application Publication
(10) Pub. No.: US 2013/0096408 A1
(43) Pub. Date: Apr. 18, 2013

(54) IMAGING EPILEPSY SOURCES FROM ELECTROPHYSIOLOGICAL MEASUREMENTS

(71) Applicants: Bin He, Arden Hills, MN (US); Lin Yang, Mesa, AZ (US)

(72) Inventors: Bin He, Arden Hills, MN (US); Lin Yang, Mesa, AZ (US)

(73) Assignee: REGENT OF THE UNIVERSITY OF MINNESOTA, St. Paul, MN (US)

(21) Appl. No.: 13/521,990

(22) PCT Filed: Jan. 13, 2011
(86) PCT No.: PCT/US11/21161

§ 371 Date: Nov. 26, 2012

Related U.S. Application Data
(60) Provisional application No. 61/335,904, filed on Jan. 13, 2010.

(51) Int. Cl. A61B 5/0476; A61B 5/00; A61B 5/0478; A61B 5/04

(52) U.S. Cl. CPC A61B 5/0476; A61B 5/04012; A61B 5/6814; A61B 5/742; A61B 5/6868; A61B 5/0478; A61B 5/04014
USPC 600/378; 600/544

(57) ABSTRACT

An example includes a method of imaging brain activity. The method includes receiving signals corresponding to neuronal activity of the brain. The signals are based on a plurality of scalp sensors (110). The method also includes decomposing the signals into spatial and temporal independent components (140). In addition, the method includes localizing a plurality of sources corresponding to the independent components. The method includes generating a spatio-temporal representation of neural activity based on the plurality of sources.

### CLAIM OF PRIORITY

This patent application claims the benefit of priority, under 35 U.S.C. Section 119(e), to Bin He et al., U.S. Provisional Patent Application Ser. No. 61/335,904, entitled “METHOD AND APPARATUS FOR IMAGING EPILEPSY SOURCES FROM ELECTROPHYSIOLOGICAL MEASUREMENTS,” filed on Jan. 13, 2010 (Attorney Docket No. 600.743PRV), which is incorporated by reference herein in its entirety.

### STATEMENT REGARDING FEDERALLY SPONSORED RESEARCH OR DEVELOPMENT

This invention was made with government support under award numbers NIH RO1 EB007920 and RO1 EB 006433 from National Institutes of Health. The government has certain rights in this invention.

### BACKGROUND

Epilepsy is a common neurological disorder affecting millions of people worldwide. In many patients, the seizures are not controlled by any available drug therapy. Partial epilepsy (seizures that begin in a focal region of the brain) represents one type of intractable epilepsy, and can be difficult to treat.

Epilepsy surgery may provide a cure, i.e. complete seizure freedom, but it is a viable option only if the brain region generating seizures can be accurately localized and safely removed. Thus, accurate localization of epileptogenic brain regions responsible for seizures is important for successful epilepsy surgery.

Seizure activity has been diagnosed using scalp electroencephalograms (EEG), using intracranial EEG (iEEG), and other modalities. Scalp EEG provides good temporal resolution but is imprecise as an imaging tool for identification of a seizure onset zone. Scalp EEG enjoys low risk and low cost relative to iEEG. See for example, Hamer H M, Morris H H, Mascha E, Bingaman W, Lüders H O. Complications of invasive video-EEG monitoring with subdural grid electrodes. Epilepsia 1999c; 40 (Suppl 7): S154. See also Rosenow F and Luders H. 2001. Presurgical Evaluation Of Epilepsy. Brain 124(9):1683-1700.

Other imaging techniques with low invasiveness are described in Blumenfeld H, Varghese G, Purcaro M, Motelow J, Enev M, McNally K, Levin A, Hirsch L, Tikofsky R, Zubal I. 2009. Cortical And Subcortical Networks In Human Secondarily Generalized Tonic-Clonic Seizures. Brain 132(4):999; Knowlton R C, Elgavish R A, Al Bartolucci B O, Limdi N, Blount J, Burneo J G, Ver Hoef L, Paige L, Faught E, Kankirawatana P. 2008. Functional Imaging: II. Prediction Of Epilepsy Surgery Outcome. Annals of Neurology 64(1):35-41; Laufs H and Duncan J S. 2007. Electroencephalography/functional MRI In Human Epilepsy: What It Currently Can And Cannot Do. Current Opinion in Neurology 20(4):417; Tyvaert L, Hawco C, Kobayashi E, LeVan P, Dubeau F, Gotman J. 2008. Different Structures Involved During Ictal And Interictal Epileptic Activity In Malformations Of Cortical Development: An EEG-fMRI study. Brain 131(8):2042-2060; and Vitikainen A M, Lioumis P, Paetau R, Salli E, Komssi S, Metsahonkala L, Paetau A, Kicic D, Blomstedt G, Valanne L, and others. 2009. Combined Use Of Non-Invasive Techniques For Improved Functional Localization For A Selected Group Of Epilepsy Surgery Candidates. Neurolmage 45(2):342-348.

Single photon emission computerized tomography (SPECT) and functional magnetic resonance imaging (fMRI) can assist in the delineation of epileptogenic brain but are also noted for their lack of temporal resolution. In addition, fMRI cannot be performed during seizure in most patients due to safety and data quality reasons.

Using EEG and MEG data, dipole source localization methods used for epilepsy source localization are limited in several aspects. For example, the number of dipole sources has to be decided a priori or some ad hoc source model has to be assumed, such as a single dipole model. Thus errors in model misspecifications may lead to errors in localization of epileptiform activity. Furthermore, the nonconvexity of the least-squares cost function normally employed using dipole source localization becomes much more severe and nonlinear multidimensional searching becomes unpractical as the number of dipoles increases. On the other hand, weighted minimum norm estimations based on the distributed current source model is underdetermined and thus necessitates the introduction of priors in order to solve the inverse problem, which typically smoothes the estimation. Many distributed source imaging algorithms can only be used at a specific time point or are limited to a short window of data. Thus, one limitation in seizure source imaging is the lack of a principled way to image epilepsy sources during seizure which can span a time duration of several seconds to several minutes.

In summary, accurate localizing of a seizure onset zone (SOZ) and dynamic imaging of epilepsy sources during ictal period for surgical intervention remains elusive.

### OVERVIEW

An example of the present subject matter includes a high-resolution EEG monitoring and dynamic source imaging approach for pre-surgical localization of SOZs and seizure propagation patterns in epilepsy patients. In addition to pre-surgical planning, the imaging results may facilitate neurosurgical treatment of medically intractable epilepsy, or guide rationale neuromodulation strategies for reducing seizures or preventing seizures from occurring. One example of the present subject matter includes a dynamic source imaging method that can be used to image other types of continuous rhythmic activity during normal brain functions or brain disorders.

In contrast with the present subject matter, some electroencephalograms/magnetoencephalograms (EEG/MEG) studies have focused on source imaging of interictal spikes and short epileptiform discharges as opposed to ictal periods. However, the precise correlation of the source of these interictal events and the clinical SOZ remains unclear. Interictal EEG spikes identify the region of electrographic seizure onset in some, but not all, pediatric epilepsy patients. Epilepsia 51(4):592-601.

Reliable recording of seizure data can entail prolonged monitoring of patients for multiple days in conjunction with suitable methods for imaging the dynamic ictal process.

An example of the present subject matter provides a dynamic process based on non-invasive EEG data (time-variant, spatial-variant, and frequency-variant); dense-array EEG/MEG sensors (e.g., 76-electrode system) and multiple-day monitoring (5.5±3.2 days). In addition, the present subject matter includes a method for identifying ictal activity with good correlation with iEEG and surgical outcomes.

One example entails using high-resolution video EEG monitoring (5.5±3.2 days) using 76 individual electrodes glued over the scalp according to a modified 10-20 montage. The EEG recordings can be referenced to CPz, passed through a 1-70 Hz bandpass filter, and sampled at 500 Hz.

An example includes a method of imaging brain activity. The method includes receiving signals corresponding to neuronal activity of the brain. The signals are based on a plurality of scalp sensors. The method also includes decomposing the signals into spatial and temporal independent components. In addition, the method includes localizing a plurality of sources corresponding to the independent components. The method includes generating a spatio-temporal representation of the whole brain neural activity based on the plurality of sources. In one such example, the scalp sensor can include EEG electrodes recording EEG. The sensors can also be MEG sensors recording MEG.

Of clinical interest is to probe the sources underlying the seizure activities, which are considered to be more reliable than interictal spikes in localizing the epileptogenic foci. The interictal activity is normally of spike shape in time domain, which allows performing source analysis at each instant during the spike. Reversely, the ictal activity is naturally a time evolving process, which requires that source analysis approaches must be able to handle spatial and temporal information simultaneously and synthetically. For this reason, few studies have addressed ictal source localization, in comparison with the interictal source localization. In addition to the spatio-temporal dipole model for ictal source localization, some other investigators combined the frequency analysis and source localization analysis to reconstruct sources from spatial pattern for certain frequency component of the ictal rhythm. The temporal segmentation of ictal rhythms, which divided activities in time domain into short time windows or a series of “functional microstate”, was also proposed in which each microstate was stable within its time window. The source localization was then achieved using mean potential map from a microstate. Due to the current lack of understanding on seizure mechanisms, such “ad hoc” selection of a certain frequency component or a “microstate” from ictal data may not lead to accurate source localization since only a portion of the information is extracted without sound justification.

As seizure activities represent an evolution of ictal rhythmic activity of the epileptic brain, an innovative way of imaging the evolution of oscillatory brain activity is needed in order to image seizure sources.

Similar to EEG source localization, MEG has been used to localize and image epileptiform activity. Due to the difficulty in seizure recordings, MEG has been used to image epilepsy sources during interictal spikes or for absence seizures (when there are no movements). Thus for the majority of seizure patients, MEG currently does not offer direct capability of recording and imaging of seizures. Even assuming successful recording of ictal MEG, the lack of rationale algorithms to image seizure sources applies to MEG recordings as well.

An example of the present subject matter includes a technique for imaging epileptogenic brain activity during seizures. One example of the present subject matter integrates the EEG inverse solution with the independent component analysis (ICA). The EEG inverse solution can include a 3-dimensional linear inverse solution, a cortical source linear inverse solution, a nonlinear inverse solution, a sub-space scanning inverse solution, a dipole localization solution, or any other inverse solution to image the sources from EEG (or other) measurements. The source separation technique may include ICA, principal component analysis (PCA), or any other blind source separation (BSS) method to separate a mixed spatiotemporal signal into a series of components.

One example includes an ictal spatiotemporal source imaging technique which involves blind source separation (BSS) in the sensor space followed by source analysis of separated spatial features of each independent source and source recombination in the source space. This example allows analysis of the seizure activity in separated time and space domains with minimal mutual interference from other activated regions and provides a whole brain spatiotemporal scan of seizure activities.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram depicting spatio-temporal seizure source imaging, according to one example.

FIGS. 2A and 2B illustrate decomposed data in a sensor domain corresponding to seizures for a representative patient.

FIGS. 3A and 3B illustrate imaging in a source domain corresponding to spatial localization of seizure onset zones and seizure propagation, and temporal reconstruction of source wave forms for selected patients.

FIG. 4 illustrates a system according to one example.

### DETAILED DESCRIPTION

An example of the present subject matter provides a dynamic seizure imaging (DSI) approach based upon high-density EEG recordings. An example can be used to image the dynamic changes of ictal rhythmic activity or discharges that evolve through time, space and frequency. In one example, the data can be generated using non-invasive sensors or generated using one or more invasive sensors.

According to one example, the method provides dynamic imaging of ictal rhythmic activity for a time before seizure onset, during seizure onset, and after seizure onset. For example, the time can be segmented to provide ictal epochs of approximately 30 seconds before and following the seizure onsets. The window length for each epoch can be varied to avoid moving artifacts, and also to include a period of background signal before seizure onset and a period of highly synchronous seizure activity following the onset. The window length can also be tailored to any time period of interest.

According to one example, the realistically shaped multi-layer boundary element model (BEM) constructed from pre-operative MRI images can be used in the seizure source imaging. The head volume can be separated into multiple conductivity layers of the brain, the skull and the scalp, and/or CSF. Other head conductor models may also be used including the finite element model, finite difference model or spherical models. To achieve accuracy to guide neurosurgical planning, realistic geometry head models may be used.

A 3-dimensional (3D) distributed source model can be used, where a number of current dipoles with unconstrained orientations can be positioned within the brain volume or occupy the gray matter or the brain volume. In one example, a cortical current source model, where a number of current dipoles with either unconstrained orientations or oriented perpendicular to the cortical surface, is used. The number of dipoles may be in the range of 5000-10,000. Alternatively, multiple dipoles source models may also be used with each representing one focused area of brain activity.

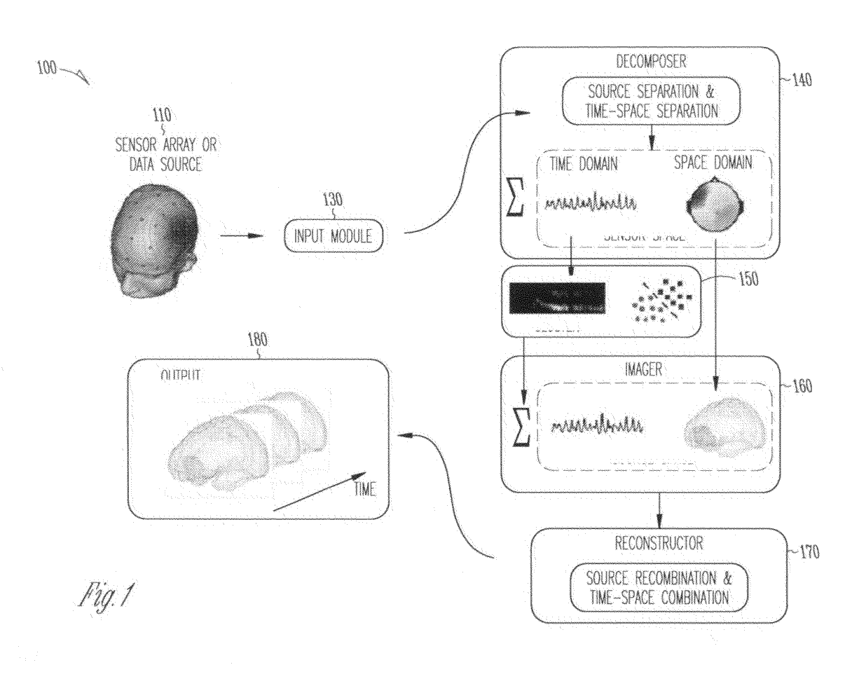

FIG. 1 illustrates a schematic for implementing an example method. The figure illustrates system 100 configured to disentangle seizure components from ictal EEG data, localization and imaging of neural generators of seizure components, and recombination of all the seizure generators in 3D brain source space to form spatiotemporal imaging of the seizure activity. The example shown is suitable for imaging continuous rhythmic activity.

The example shown can be used with data provided by prolonged multiple electrodes video EEG monitoring. The spatiotemporal seizure imaging technique illustrated is based on BSS in the sensor space, as shown at in the figure. In addition the method includes source analysis performed separately in the time domain and the space domain. Furthermore, the method shown includes source recombination and time-space re-combination in the source domain. The reconstructed seizure activities compares favorably with other clinical evidence, including surgically resected regions, iEEG recording, SPECT, and successful surgical outcome.

System 100 can include a processor, circuitry, and other systems to implement the methods described herein. Sensor array or data source 110 can include multiple sensors or, in one example, can include stored data corresponding to electrical activity of a biological system (such as a brain). Input module 130 can include an interface is configured to receive data or signals from sensor array or data source 110. Input module 130 provides data to decomposer 140. Decomposer 140 performs a separation algorithm and in one example, this includes source separation and time-space separation. Cluster module 150 is configured to select particular components (provided by decomposer 140) of interest for further analysis. Imager 160 is configured to identify a location of a component in the source space. Reconstructor 170 is configured to reconstruct a dynamic source model based on the data provided by imager 170. Reconstructor 170 provides an output to output module 180 which is configured to render an spatio-temporal representation of the electrical activity. In one example, output module 180 includes a display.

In the EEG forward model, the spatiotemporal EEG scalp recording Y can be related with underlying brain activity S through a linear system:

Y=LS+B (Equation 1)

Where Y( r̅, t̅) is a n*t signal matrix (n is the number of electrodes and t is the number of time points), S( r̅, t̅) is a m*t source matrix (m is the dimension of source space) and B is a n*t noise matrix. L is a n*m lead field matrix that can be calculated based on the boundary element method (BEM) (Fuchs et al., 1998; Hamalainen and Sarvas 1989; He et al., 1987) or based on a finite element method, a finite difference method, or another numerical method. In the BEM model, the head volume conductor can be separated into three conductive layers, the brain, the skull and the skin with conductivity of 0.33 S/m, 0.0165 S/m and 0.33 S/m, respectively (Lai et al., 2005; Oostendorp et al., 2002; Zhang et al., 2006). In one example, the BEM model can be separated into four conductive layers, the brain, the skull, the skin and the CSF. A 3D distributed source model can be used to model the brain source distribution that includes around ten thousand equivalent current dipoles with unconstrained orientations evenly positioned within the 3D brain volume. In one example, a cortical current model (CCD) that constraints the dipoles within the cortical sheet of gray matter and multiple dipoles source models can be used.

Electrode positions in a modified 10-20 system can be used for the calculation.

Ictal EEG measures seizure rhythmic discharges that evolve through time, space, and frequency, superposed with measurement noise, moving artifacts and other background brain oscillations. To analyze such complicated signal, independent component analysis (ICA) can be used to decompose each ictal EEG into a series of temporally independent and spatially fixed components:

$$Y = WQT = {\sum }_{i = 1}^{Nc} {w}_{i} {Q}_{i} {T}_{i} \qquad ( Equation 2 )$$

where N_{c} is the number of ICs, Qᵢ (i^{th} column of the matrix QₙN_{c} is the spatial map of the i^{th} IC, Tᵢ (i^{th} row of matrix T_{Nc*i}) is the temporal dynamics of the i^{th} IC, and W is a diagonal weighting matrix. ICI is but one example and other BSS techniques can be used for the decomposition of the signals (EEG, MEG, or other) Assuming Nₛ out of the Nc ICs are associated with seizure activities (component selection presented later), the scalp measurement generated by ictal conditions becomes

$$Y = WQT = {\sum }_{i = 1}^{Ns} {w}_{i} {Q}_{i} {T}_{i} .$$

Given the forward modeling of lead field matrix, spatiotemporal brain sources can be estimated from the EEG measurements by solving an inverse problem as follows (Pascual-Marqui et al., 1994):

Ŝ=L⁻¹Y (Equation 3)

where L⁻¹ is the inverse of lead field matrix. Substituting equation 2 into equation 3, the spatiotemporal estimation can be rewritten as:

$${S}^{⋒} = {L}^{- 1} {\sum }_{i = 1}^{Ns} {w}_{i } {Q}_{i} {T}_{i} = {\sum }_{i = 1}^{Ns} [ {L}^{- 1} {Q}_{i} ] {w}_{i} {T}_{i} = {\sum }_{i = 1}^{Ns} {{S}^{⋒}}_{i} {w}_{i} {T}_{i} \qquad ( Equation 4 )$$

where Ŝᵢ=L⁻¹Qᵢ is the IC source distribution of the i^{th} IC, and

$${\sum }_{i = 1}^{Ns} {{S}^{⋒}}_{i} {w}_{i} {T}_{i}$$

is the linear combination of seizure components in the source space, which can be seen as an inverse process of ICA. Here, an algorithm known as Low Resolution Electromagnetic Tomography (LORETA) (Pascual-Marqui et al., 1994) can be used to estimate Ŝᵢ of each seizure component. Other EEG/MEG distributed imaging algorithms, such as minimum norm estimate (MNE), variants of MNE (e.g., weighted MNE), L-p norm algorithms (e.g., L-1 norm), sub-space scanning algorithms such as MUSIC, RAP-MUSIC, FINE algorithms, or dipole source localization algorithms can be incorporated into this method to estimate Ŝᵢ of each seizure component. Given the reconstructed dynamic source signal Ŝ, the SOZ can be identified as the source distribution at the seizure onset time instant. Similarly, the time-variant propagation of seizure activity over the prolonged ictal period can also be estimated and visualized during a time window after the seizure onset.

### EXAMPLES OF A COMPONENT SELECTION METHOD

Seizure activities are characterized by abnormal synchrony of neuronal rhythmic discharges. Time-frequency evolution patterns of ictal rhythmic discharges are observable in raw EEG recordings and also in ICs related with ictal conditions. As such, the time-frequency similarity between the two signals can be used for the selection of seizure components.

In each seizure recording, visual inspection can be used to remove those ICs showing continuous activity or transit spikes (e.g., in IC time courses or spectrograms) not correlated with seizure conditions, such as the eye movement components (which may show IC spatial maps with frontal eye activity) and moving artifactual components (which may show strong power invariant across all the frequency bands, and/or with spatial maps dominated by noise. The noise-deducted EEG can then be reconstructed. Electrodes can be identified by epileptologists that show ictal rhythmic discharges and calculate the mean time frequency representation (TFR) of EEG recorded by these electrodes (EEG-TFR) using short time Fourier transformation (sliding window size 500 time points, 50% overlapping). In one example, the spectrogram TFR can be calculated using other techniques, such as a wavelet-based algorithm.

A TFR can be computed for each IC (including all the ICs derived from ICA). Correlations between each IC-TFR and EEG-TFR can be calculated. Statistical significance of the correlation between EEG-TFR and each IC-TFR can be quantified by a nonparametric statistical test technique using a surrogate method. In one example, surrogate datasets can be created from EEG signal and each IC time course so that their mutual correlations are not preserved. For each IC, new correlations between EEG-TFRs and IC-TFRs can be computed from surrogated datasets and a distribution of correlation values can be obtained. From the distribution, statistical significance of an IC-EEG-correlation can be decided and those components exceeding a threshold of p=0.1 can be selected as seizure components for further source analysis. Visual inspections can also be used to assist the selection of seizure components.

In one example, component selection includes the implementation of a clustering technique. For each seizure, the independent component—time frequency representation (IC-TFR) and the EEG-TFR can be analyzed by K-means clustering. Each of these can be treated as a point in the space and the distance function is defined by:

d=1−r_{corr}

where r_{corr} is the correlation between the points. These TFR points can be partitioned into groups by minimizing the within-group sums of the point-to-cluster-centroid distances. Those ICs in the same group of EEG-TFR represented time-frequency features closely relate to the ictal rhythms and therefore can be selected as the seizure components for the subsequent source analysis. In one example, other clustering algorithms are used to select components of interest for further analysis. Clustering is performed, in one example, by cluster module 150 shown in FIG. 1.

According to one example, a time frequency representation (TFR) can be calculated from the raw EEG data by convolving the signal with complex Morlet's wavelets. The time-frequency evolution of the ictal rhythm can be tracked by EEG-TFR. Similarly, TFR can also be calculated from the time courses of each IC to examine the time-frequency features of each component. Those ICs related with eye, severe muscle and electrode artifacts can be removed by examining their spatial and time-frequency features.

### EXAMPLE OF METHOD TO IMAGE THE SEIZURE ONSET ZONE AND PROPAGATION

The spatiotemporal imaging output has whole-brain coverage, high temporal resolution (millisecond for EEG and MEG) and high spatial resolution (depending on the resolution of the head model). Source waveforms can be reconstructed from any regions of interest of the brain. Further analysis, such as time-frequency analysis, coherent and connectivity analysis based on the waveforms at individual source locations or regions of interest can be conducted.

The determination of the SOZ is used in epilepsy surgery. An example of the present subject matter can first define the SOZ as the source distribution at onset time instant. Intracranial EEG directly recorded from the cortex and brain surgery outcomes can be used to quantitatively evaluate the performance of the SOZ localization. In addition to determining the SOZ, an example of the present subject matter can be used to reconstruct continuous propagation patterns of ictal rhythmic activity as source distributions at instants after the seizure onset. Continbous source wave form can be achieved in a voxel of interest or a region of interest. Source time-frequency features can be reconstructed in the 3D source space. The time-varying source power in each brain voxel can be calculated as the spectral power within the predominant frequency band of ictal rhythm during short time intervals. The source power distribution over the ictal period can indicate the propagation of ictal rhythmic discharges from a focal location to extended regions.

### EXAMPLES OF IDENTIFICATION OF ICTAL COMPONENTS

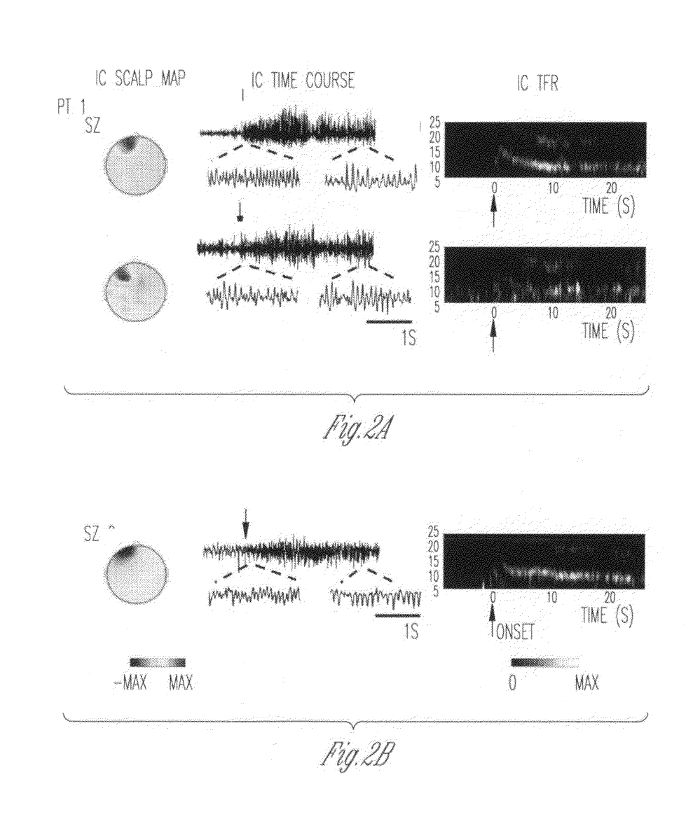

Using ICA and component selection, multiple ICs can be identified from each seizure to represent ictal activity, as shown for example, with regard to the sample patient depicted in FIGS. 2A and 2B. In the figures, the patient data illustrate frontal lobe epilepsy. Using ICA, two components can be identified from one seizure (FIG. 2A). The IC time courses show ictal rhythms having increased frequency at seizure onset (near the vertical arrows) and decreased frequency in the alpha band at a later time. A is time bar is illustrated in the figure. The IC-TFRs also show increased neural synchrony initiated with fast rhythmic activity >12 Hz at the seizure onset that later progress to an alpha frequency discharge. In the IC-TFR representations, the vertical scale is in Hz with legends depicting 0, 10, 15, 20, and 25 Hz and the horizontal scale depicts time with legends at 0, 10, and 20 seconds. This time-frequency evolution pattern of the ictal rhythmic discharges is consistent with independent observation reported by clinical epileptologists. The corresponding scalp map shows left frontal focus of the seizure components with some spread to the temporal lobe in the second seizure illustrated. The two seizures recorded in this patient (and shown in the figure) illustrate similar EEG rhythmic discharges. One component identified from another seizure of the patient (shown in FIG. 2B) shows similar rhythmic discharges at the seizure onset. The corresponding scalp map further localized this seizure component to the left frontal electrodes.

In addition to determining the SOZ, an example of the present subject matter can be used to reconstruct propagated activity after seizure onset. Time-varying source power in each brain voxel can be calculated as the spectral power within the predominant frequency band of ictal rhythm during short time intervals. The source power distribution over the ictal period can indicate the propagation of seizure activity from a focal location to extended regions ipsilaterally or contralaterally. The source power can be calculated based on a short time window and some spread of the source distribution to adjacent cortex, such as the area of activation in frontal cortex of a patient may be observed in some instances. Also, source time-frequency features can be reconstructed in the 3D source space and used to display TFRs of the SOZ tissue and show the time-frequency features of each seizure.

The ICA can be used to provide a method to separate seizure components from continuous EEG recordings. To further determine the spatiotemporal activation patterns of ictal activities within the whole brain, the ICs' source map and time courses can be combined into the 3D brain source space as an inverse process of ICA. The estimated SOZ is defined as the source distribution at the seizure onset time.

Recordings from numerous scalp sites can provide a good spatial sampling rate and a stable spatial representation.

EEG monitoring can entail a 19-to-32-electrode montage or can include a high-resolution EEG. Increasing the channel number to 76 provides more spatial detail for the localization of epileptic sources. In various examples, the number of electrodes (channels) can be 19, 31, 32, 63, 123, or greater, or can be in any range between these numbers.

Most clinical applications of high-resolution EEG and MEG have been restricted to the imaging of interictal spikes, despite the fact that the irritative zone defined by interictal spikes do not reliably determine the minimum region of brain tissue to be resected in order to render the patient seizure free. A technical limitation lies in the recording environment where the patients are to stay motionless in order to obtain high-quality signals. This is especially true for MEG recordings.

An example of the present subject matter provides a method of seizure imaging to reconstruct dynamic ictal rhythmic discharges from continuous EEG data. Fitting single or multiple dipoles to the early activation of ictal rhythms has been demonstrated as useful in providing sublobar prediction of seizure origin in temporal lobe seizures and extra-temporal lobe seizures. However, such methods rely on prior information such as the number of dipoles or the positions of dipoles which cannot be easily gained from EEG signal alone. Sub-space scanning methods also provide the ability to reconstruct temporal dynamics of seizure sources, and, in conjunction with connectivity analysis, may be able to discriminate the seizure onset and propagation. In these situations, however, the limited number of equivalent dipoles (as discrete sources) may not be an appropriate representation of the distributed brain activity involved in seizures. Assuming a distributed nature of seizure activity, one method entails judging the seizure onset by visual inspection of EEG waveforms and then conducting source imaging instant by instant to find neural generators responsible for each millisecond or for each short time window. Such a process may require solving thousands of inverse problems in order to achieve several-second-long source imaging.

Also, low SNR at the time of seizure onset adds a level of complexity for the disentanglement of seizure source, physiological noise and artifactual noise.

An example of the present subject matter entails a dynamic source imaging technique that is particularly suited for continuous imaging of seizure activity expanding from several seconds to several minutes. This dynamic spatiotemporal imaging approach entails a decomposition-recombination process, where the decomposition is taken in the sensor space and the recombination is taken in the source space. Such a process, regardless of the length of continuous seizure data, limits the number (equal to the number of selected seizure components) of inverse problems to be solved. Additionally, the separation of ictal components from artifacts, noises and other background brain oscillations largely enhances the SNR for the source analysis. This approach can be also seen as a time-space-separated process.

The data-driven ICA analysis decomposes the signal into several spatially fixed but temporally dynamic components. In the time domain, the time-frequency evolution represented in the time course assists in the selection of the seizure components. This approach to component selection allows for the extraction and imaging of certain rhythmic modulation (e.g., delta rhythm that may later progress to theta rhythm), and thus is well suited for analysis of time-varying ictal rhythmic activity.

### EXAMPLES OF LOCALIZATION OF SEIZURE ONSET ZONES AND PROPAGATION

According to experimental results, the locations and extensions of the estimated SOZs shows good agreement with the epileptogenic zone resected in surgery or defined by iEEG invasive measurements.

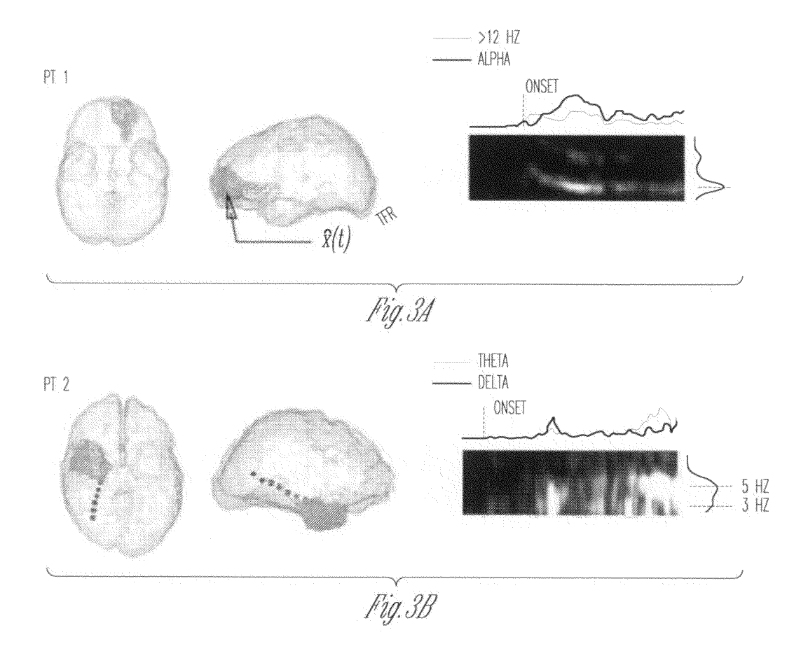

FIGS. 3A and 3B each illustrates the estimated SOZs and the source TFRs estimated from typical seizures in each of two patients. The estimated SOZs are shown as darker regions in the left and middle panels. The two patients were both rendered seizure-free after surgery and one-year follow-up. The surgically resected regions are depicted. Intracranial electrodes were implanted in patient 2 (FIG. 3B; shown at a location using spherical dots) and the anterior electrodes (marked) were defined by clinical epileptologists as the seizure onset zone. As illustrated in FIGS. 3A and 3B, the estimated SOZ in each of the patients is co-localized with the surgically resected region and also the direct measurement from intracranial electrodes. The figures also illustrate the continuous imaging of the two seizures, which start from epileptogenic cortex and later propagate to adjacent lobes. The time-frequency analysis of the estimated source waveforms at the seizure onset zone depicts the dynamic evolution of ictal rhythmic activity that changes in time and frequency.

### EXAMPLE SYSTEM

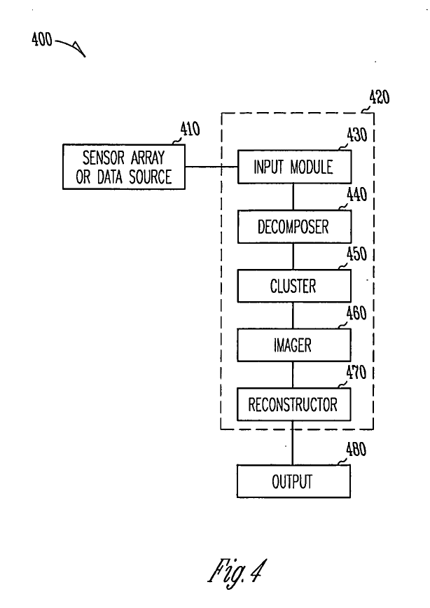

FIG. 4 illustrates system 400 according to one example. As shown in the figure, system 400 includes sensor array or data source 410. In the form of a sensor array, this can include a grid or electrode assembly having any number of discrete sensors. For example, this can include scalp EEG sensor, intracranial EEG sensors, MEG sensors, or other type of sensors configured to detect neuronal activity. In one example, neuronal data is stored in a memory device and as such, the memory device serves as data source 410.

Sensor array or data source 410 is coupled to apparatus 420. Apparatus 420 can include one or more processors (digital or analog) configured to implement an algorithm or otherwise perform a function as shown or described herein.

Input module 430, of apparatus 420 can include an interface to receive a signal or data from sensor array or data source 410. Input module 430 can be configured to receive an analog signal or digitally encoded data. Input module 430 is coupled to sensor array or data source 410.

Decomposer module 440 can be viewed as a second module and, in one example, is configured to decompose a signal (or a plurality of signals) into individual components. In one example, decomposer module 440 implements an algorithm known as Independent Component Analysis (ICA) based on the signals from the sensor array 410. Other signal separation techniques that realize the separation of spatiotemporal signals into components each of which is represented by a time course and a spatial map can be readily incorporated in decomposer module 440 to replace ICA. Examples of the signal separation techniques include principal component analysis (PCA), other forms of ICA, or any of which belong to a class of techniques more generally described as blind source separation (BSS). Decomposer module 440 is coupled to input module 430.

Cluster module 450 can be viewed as the third module, and in one example, is configured to select components of interest for further analysis. In one example, the selection of seizure components in decomposer module 440 is implemented by calculating the correlation between the spectrograms of independent components and spectrograms of original EEG signals. The statistical significance of the correlation is tested using surrogate data. In another example, the selection of seizure components is implemented by k-means clustering that cluster the spectrograms of components into several subsets. Other methods that select the components with temporal features of interest, frequency features of interest, or spatial patterns of interest can be readily incorporated to choose components for the input of the next module. Examples include visual inspection of the waveforms and the spatial maps, and various types of clustering techniques. If PCA is used for decomposition, the rejection of components of small eigenvalues also serves this purpose. If the system is applied to image brain activity in well-designed experiments, components can be also selected based on prior knowledge, and certain modulation patterns corresponding to the behavior in neuroscience studies. Cluster module 450 is coupled to decomposer module 440.

Imager module 460 can be viewed as a fourth module and, in one example, is configured to determine the location of a component within a source space. Imager module 460, in one example, implements a BEM head model, a 3D distributed source model and a source estimation algorithm. Other head models can also be implemented, including a spherical head model, a finite element model (FEM), and a finite difference model. Other source models including cortical current density (CCD) model, and equivalent dipole models can also be implemented. Other algorithms solving inverse problems can also be implemented, including minimum norm estimate (MNE), variants of MNE (e.g. weighted MNE), non-linear techniques based on L-p norm (p<2) (e.g., L-1 norm), any of which belong to a class of techniques more generally described as distributed source imaging. The method can be also generalized to include sub-space scanning algorithms such as MUSIC, RAP-MUSIC, FINE, and nonlinear source estimation algorithms such as equivalent dipoles or more complicated source models based on brain networks. Imager module 460 is coupled to cluster module 450.

Reconstructor module 470 can be viewed as a fifth module and, in one example, is configured to reconstruct a dynamic source signal (or a plurality of signals) based on the estimation of source components from imager module 460 and time courses of components from decomposer model 440. This module combines the signal in the source space, which can be seen as an inverse process of the decomposer module. In one example, reconstructor module 470 implements a linear combination which sums the components' time courses weighted by the components source distribution. Variants of the components' time courses can also be input into the reconstructor module, such as certain frequency bands of the time courses and the temporal modulation of the spectral power. Reconstructor module 470 provides the spatiotemporal imaging involving all the components of interest. It results in a continuous imaging of the whole brain with high spatial resolution and high temporal resolution. Reconstructor module 470 is coupled to imager module 460.

Reconstructor module 470 is coupled to output module 480. Output module 480 can include a display, a memory device, or a network interface device. In one example, output module 480 implements the visualization of the seizure onset zone (SOZ) at the onset of the seizure, and the seizure propagation pattern after the onset of the seizure. In one example, output module 480 implements the visualization of the temporal dynamics and time-frequency spectrogram from a voxel in the source space. Output module 480 provides the spatiotemporal brain imaging of a continuous period with high temporal resolution (e.g., millisecond for EEG, iEEG and MEG). Various types of analysis based on the source spatial information or temporal information can be incorporated into the output module 480. Examples include the display of movie of source activity over any period of time, the visualization of source distribution of different frequency bands, the localization of source activity at any time instants or any time intervals, connectivity or coherent analysis across various regions of the brain. In various examples, system 400 provides a user-perceivable output corresponding to the neuronal activity of the brain.

Other modules and components can also be included in system 400. For example, apparatus 420 can include an additional memory device (such as a user-replaceable storage device), or a telemetry device configured to wirelessly communicate data, results, or instructions.

A functional MRI (fMRI) module can also be implemented by apparatus 420 and, in one example, is located between module 440 and module 460. A fMRI module uses the components' time courses' to image fMRI map through EEG-informed fMRI analysis and use the fMRI maps to constrain the source localization in imager module 460 through fMRI-weighted EEG source imaging analysis. Also, the component selection method disclosed here (correlation of spectrograms of IC and EEG and subsequent statistical analysis), although shown to be part of the system 400, can be readily applied in other methods to identify ictal rhythmic discharges. The present subject matter can be applied to imaging of seizure activity as well as for the imaging of any type of continuous brain activity in any experimental settings, for example, interictal activity and background oscillation of patients during resting state, modulation of continuous rhythmic activity in healthy subjects, or any other oscillatory brain activity in healthy subjects or patients with any other neurological disorders or psychiatric diseases.

### EXAMPLE FOR IMAGING CARDIAC ELECTRICAL ACTIVITY

An example of the present subject matter can be applied to image cardiac electrical activity from electrocardiogram (ECG), magnetocardiogram (MECG), or intracavitory electrophysiological recordings. In such an example, multiple channels of ECG/MCG or intracavitory recordings are decomposed into temporal and spatial components. Inverse solutions are then solved to estimate the cardiac electrical sources corresponding to the independent components using a linear or nonlinear inverse solution. The inverse solutions of independent components are then recombined in the source domain to form the spatio-temporal representation of source distribution of a heart.

An example can also be used to localize and image origins and propagation of cardiac arrhythmias from body surface ECG signals or from intracavitory recordings such as using a catheter.

### ADDITIONAL NOTES AND EXAMPLES

Examples of the present subject matter can be used for long-term monitoring (using dense-array EEG sensors), used to localize a SOZ or image functional networks involved in seizure initiation and propagation for pre-surgical and surgical planning. One example enables dynamic imaging to trace propagation of seizure activity. For example, one embodiment allows spatio-temporal source imaging of brain activity including continuous ictal rhythmic discharges.

The present subject matter can be applied to imaging and localizing epileptogenic brain and epileptic propagation to aid presurgical and surgical planning for treatment of epilepsy patients. An example of the present subject matter can be used to estimate seizure sources from either EEG or MEG recordings or iEEG.

Seizure activity can be an oscillatory activity evolving over time. Conventional techniques can only be applied to a time point or a small segment in time, ignoring the temporal evolving nature of seizure. The present subject matter provides a rigorous means to extract the spatio-temporal source distribution of ictal rhythm.

High-resolution EEG can be used as a pre-surgical imaging tool which provides additional information about the precise location and extent of the SOZ and without the additional costs and risks associated with iEEG. In one example, iEEG grids or electrodes are positioned at the most suspicious regions, which are decided by prior knowledge gained from scalp EEG.

Various examples and implementations can be provided based on the present subject matter. For example, the present subject matter can be used for spatiotemporal imaging of continuous ictal rhythmic discharges with high resolution. In addition, the present subject matter can be used for long-term monitoring of seizure using dense-array EEG recording in epilepsy patients. One example can be configured to provide localization of a SOZ for presurgical planning of epilepsy treatment. In addition, one example provides dynamic imaging tracing of the propagation of seizure activity. Furthermore, one example provides spatio-temporal source imaging of rhythmic brain activity.

An example of the present subject matter may be useful in managing epilepsy by means of neuromodulation. Knowledge of epileptogenic brain can provide useful information to optimize the neuromodulation strategies for reducing or preventing seizures from occurring.

One example provides epilepsy source information to aid neuromodulation to reduce or prevent seizures from occurring.

Example 1 includes a method of imaging brain electrical activity and includes collecting signals over a part of the head or over a part of a surface out of the head using a plurality of sensors and a data acquisition unit. The method also includes decomposing the collected multi-channel signals onto a series of spatial and temporal independent components using Independent Component Analysis. In addition, the method includes constructing a source distribution corresponding to the electrical activities of the brain and estimating the individual source distribution for the selected spatial independent components. Total brain source distribution can be reconstructed by integrating the estimated sources for the selected spatial independent components with the temporal independent components and displaying the estimated brain electrical source distributions within the three dimension space of the brain.

Example 2 includes the method of Example 1 optionally including wherein the signals are collected during an epilepsy seizure.

Example 3 includes the method of one or any combination of Examples 1-2 and optionally including wherein the signals are collected during interictal periods, including spikes or non-spike interictal periods.

Example 4 includes the method of one or any combination of Examples 1-3 and optionally including wherein the signals are collected using an array of scalp EEG electrodes.

Example 5 includes the method of one or any combination of Examples 1-4 and optionally including wherein the signals are collected using an array of MEG sensors.

Example 6 includes the method of one or any combination of Examples 1- 5 and optionally including wherein the signals are collected using an array of

EEG electrodes and MEG sensors.

Example 7 includes the method of one or any combination of Examples 1-6 and optionally further including using the estimated brain electrical sources are used to aid presurgical or surgical planning in an epilepsy patient. Example 8 includes the method of one or any combination of Examples 1-7 and optionally further including using the estimated brain electrical sources to aid neuromodulation treatment in an epilepsy patient.

Example 9 includes the method of one or any combination of Examples 1-8 and optionally wherein the independent components are selected by comparing the time-frequency representation of the temporal independent components with the time-frequency representation of the raw signals.

Example 10 includes the method of one or any combination of Examples 1-9 and optionally wherein the signals are collected using an array of intracranial electrodes.

Example 11 includes an apparatus for imaging brain electrical activity, the apparatus comprising a plurality of sensors for decomposing collected multi-channel signals onto a series of spatial and temporal independent components using ICA, a first module configured to construct a source distribution representing the electrical activities of the brain, a second module configured to estimate the individual source distribution for the selected spatial independent components, a third module configured to reconstruct the total brain source distribution by integrating the estimated sources for the selected spatial independent components with the temporal independent components, and an output module configured to display the estimated brain electrical source distributions within a three dimension space of the brain.

In one example, a system includes a plurality of sensors for collecting multi-channel signals, a first module configured to decompose multi-channel signal onto a series of spatial and temporal independent components using ICA, a second module configured to select components of interest, a third module configured to estimate the individual source distribution for the spatial maps of selected independent components, a fourth module configured to reconstruct the total brain source distribution by integrating the estimated sources with the time course of independent components, and an output module configured to display the estimated brain electrical source distributions within a three dimension space of the brain.

Example 12 includes the apparatus of Example 11 wherein the signals are collected during epilepsy seizure.

Example 13 includes the apparatus of one or any combination of Examples 11-12 and optionally wherein the signals are collected during interictal periods, including spikes or non-spike interictal periods.

Example 14 includes the apparatus of one or any combination of Examples 11-13 and optionally wherein the signals are collected using an array of scalp EEG electrodes.

Example 15 includes the apparatus of one or any combination of Examples 11-14 and optionally wherein the signals are collected using an array of MEG sensors.

Example 16 includes the apparatus of one or any combination of Examples 11-15 and optionally wherein the signals are collected using an array of EEG electrodes and MEG sensors.

Example 17 includes the apparatus of one or any combination of Examples 11-16 and optionally wherein the estimated brain electrical sources are used to aid presurgical or surgical planning in epilepsy patients.

Example 18 includes the apparatus of one or any combination of Examples 11-17 and optionally wherein the estimated brain electrical sources are used to aid neuromodulation treatment in epilepsy patients.

Example 19 includes the apparatus of one or any combination of Examples 11-18 and optionally wherein the independent components are selected by comparing the time-frequency representation of the temporal independent components with the time-frequency representation of the raw signals.

Example 20 includes the apparatus of one or any combination of Examples 11-19 and optionally wherein the signals are collected using an array of intracranial electrodes.

Example 21 includes a method of imaging brain activity. The method includes receiving signals corresponding to neuronal activity of a brain. The signals are based on a plurality of scalp sensors. The method includes decomposing the signals into spatial and temporal independent components. The method includes localizing a plurality of sources corresponding to the independent components. The method includes generating a spatio-temporal representation of neural activity based on the plurality of sources.

Example 22 includes the method of Example 21 wherein receiving signals includes at least one of receiving MEG data or receiving EEG data.

Example 23 includes the method of any of Examples 21-22 wherein decomposing the signals includes executing an independent component analysis.

Example 24 includes the method of any of Examples 21-23 wherein localizing the plurality of sources includes estimating a source distribution using the independent components.

Example 25 includes the method of any of Examples 21-24 wherein localizing the plurality of sources includes generating a time-frequency representation of EEG data or generating a time-frequency representation of data corresponding to an independent component.

Example 26 includes the method of any of Examples 21-25 wherein generating the spatio-temporal representation includes displaying source distribution within a three dimensional space of the brain.

Example 27 includes the method of any of Examples 21-26 further including selecting a surgical intervention site based on the spatio-temporal representation.

Example 28 includes a system for analyzing neural activity of a brain. The system includes an input module configured to receive data corresponding to a plurality of signals based on the neural activity. The system includes a first module configured to decompose the data into independent components. The system includes a second module configured to localize a plurality of sources corresponding to the independent components. The system includes a third module configured to generate a spatio-temporal representation of neural activity based on the plurality of sources. In one example, the system includes a second module configured to select seizure components and includes a third module configured to localize a plurality of source and a fourth module configured to generate a spatio-temporal representation.

Example 29 includes a system of Example 28 wherein the input module is configured to couple with a high density array of scalp sensors.

Example 30 includes the system of Example 29 wherein the scalp sensors include at least one of an EEG sensor or a MEG sensor.

Example 31 includes the system of any of Examples 28-30 wherein the input module is configured to couple with an intracranial electrode.

Example 32 includes the system of any of Examples 28-31 wherein the first module includes a processor configured to implement an independent component analysis algorithm.

Example 33 includes the system of any of Examples 28-32 wherein the second module includes a processor configured to evaluate an inverse problem based on the independent components.

Example 34 includes the system of any of Examples 28-33 wherein the second module includes a processor configured to implement a tomography algorithm.

Example 35 includes the system of any of Examples 28-34 wherein the third module is configured to identify a time of onset of seizure based on the spatio-temporal representation.

Example 36 includes the system of any of Examples 28-35 wherein the third module includes a display.

These examples can be combined in any permutation or combination. This overview is intended to provide an overview of subject matter of the present patent application. It is not intended to provide an exclusive or exhaustive explanation of the invention. The detailed description is included to provide further information about the present patent application.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method of imaging brain activity comprising:
receiving signals corresponding to electrical activity of a brain, the signals based on a plurality of scalp sensors;
decomposing the signals into spatial and temporal independent components;
localizing a plurality of sources corresponding to independent components selected based on spatial, temporal or spectral features of interest; and
generating a spatio-temporal representation of electrical activity based on the plurality of sources.

2. The method of claim 1 wherein receiving signals includes at least one of receiving MEG data or receiving EEG data.

3. The method of claim 1 wherein decomposing the signals includes executing an independent component analysis.

4. The method of claim 1 wherein localizing the plurality of sources includes estimating a source distribution using the independent components.

5. The method of claim 1 wherein localizing the plurality of sources includes generating a time-frequency representation of EEG data or generating a time-frequency representation of data corresponding to an independent component.

6. The method of claim 1 wherein generating the spatio-temporal representation includes displaying source distribution within a three dimensional space of the brain.

7. The method of claim 1 further including selecting a surgical intervention site based on the spatio-temporal representation.

8. A system for analyzing electrical activity of an organ, the system comprising:
an input module configured to receive data corresponding to a plurality of signals based on the electrical activity;
a first module configured to decompose the data into independent components;
a second module configured to image a plurality of sources corresponding to the independent components; and
a third module configured to generate a spatio-temporal representation of electrical activity of the organ based on the plurality of sources.

9. The system of claim 8 wherein the input module is configured to couple with a high density array of scalp sensors.

10. The system of claim 9 wherein the scalp sensors include at least one of an EEG sensor or a MEG sensor.

11. The system of claim 8 wherein the input module is configured to couple with at least one intracranial electrode.

12. The system of claim 8 wherein the first module includes a processor configured to implement an independent component analysis algorithm.

13. The system of claim 8 wherein the second module includes a processor configured to estimate a source location corresponding to the independent components.

14. The system of claim 8 wherein the second module includes a processor configured to implement a tomography imaging algorithm.

15. The system of claim 8 wherein the third module is configured to identify a time of onset of seizure based on the spatio-temporal representation.

16. The system of claim 8 further including a display coupled to the third module.
